(19) United States
(12) Patent Application Publication
(10) Pub. No.: US 2013/0197547 A1
(43) Pub. Date: Aug. 1, 2013

(54) DEVICE FOR CLOSING LUMINAL CAVITY AND METHOD THEREFOR

(71) Applicant: Terumo Kabushiki Kaisha, Tokyo (JP)

(72) Inventors: Tetsuya FUKUOKA, Shizuoka (JP); Katsunori Ebata, Kanagawa (JP)

(73) Assignee: Terumo Kabushiki Kaisha, Tokyo (JP)

(21) Appl. No.: 13/744,898

(22) Filed: Jan. 18, 2013

(30) Foreign Application Priority Data
Jan. 27, 2012 (JP) 2012-014867

(51) Int. Cl. A61B 17/122

(52) U.S. Cl. CPC A61B 17/122
USPC 606/157

(57) ABSTRACT

A luminal cavity closing device can include a flexible linear shaft, a clamping mechanism which is provided at a distal portion of the shaft and has at least two arms, and a detachment mechanism configured to enable detachment of the clamping mechanism from the shaft. The arms of the clamping mechanism can be configured so as to be insertable into hollow tubular structures located adjacent to and on both sides of a luminal cavity present in a hollow tubular structure bifurcation area. The clamping mechanism operates between an expanded state wherein the interval of the arms is enlarged to a contracted state wherein the interval of the arms is contracted.

This application claims the priority benefit under 35 U.S.C. §119 of Japanese Patent Application No. 2012-014867 filed on Jan. 27, 2012, which is hereby incorporated in its entirety by reference.

### BACKGROUND

The presently disclosed subject matter relates to a luminal cavity closing device and a method for closing a luminal cavity (e.g., for closing hollow pouched structures having a lumen with an opening, including an aneurysm or varix or the like, hereinafter generically referred to as “luminal cavity”) formed in a living organ or body.

A part of a blood vessel that is locally dilated and weakened is called an aneurysm or varix, and, particularly, one that is generated in an artery of the brain is called a cerebral aneurysm. Rupture of the cerebral aneurysm causes subarachnoid hemorrhaging. There are several therapeutic methods for preventing such a rupture. One of the methods is the “neck clipping technique” in which craniotomy is conducted neurosurgically, and the portion between the cerebral aneurysm and the parent artery (a base portion of the aneurysm) is clipped. Another of the methods is called “embolization technique,” in which treatment is carried out without craniotomy. In this method, a catheter is inserted into the cerebral aneurysm via a blood vessel, and a flexible coil formed of a metal such as platinum is guided through the catheter and embedded in the aneurysm (refer to Japanese Patent No. JP-T-2008-510594 and related English language application U.S. 2006/0200192 which are incorporated herein by reference).

### SUMMARY

The presently disclosed subject matter has been made in relation to a method for closing a luminal cavity through a lumen like blood vessel (by an intravascular interventional technique). The presently disclosed subject matter can provide a luminal cavity closing device and a luminal cavity closing method by which a rise in the internal pressure of a luminal cavity (aneurysm) can be effectively restrained and influences on the surrounding tissues can be reduced.

According to an embodiment of the presently disclosed subject matter, a luminal cavity closing device can include the following: a flexible linear shaft; a clamping mechanism which is provided at a distal portion of the shaft, has at least two arms capable of being inserted into hollow structures located adjacent to and on both sides of a luminal cavity present in a hollow structure bifurcation area, and is capable of operating from an expanded state where the interval of the arms is enlarged to a contracted state where the interval of the arms is reduced; and a detachment mechanism configured to enable detachment of the clamping mechanism from the shaft.

This configuration makes it possible to carry out a procedure for closing a luminal cavity formed in a hollow structure bifurcation area. In the procedure, first, the luminal cavity closing device is inserted into a catheter, and the clamping mechanism provided at a distal portion of the device is brought into the vicinity of the luminal cavity. Then, the luminal cavity is pressed (clamped) by the arms of the clamping mechanism through hollow structures adjacent to and on both sides of the luminal cavity. Thereafter, the clamping mechanism is detached from the shaft. With the luminal cavity thus pressed through the hollow structures on both sides, the opening (aperture) of the luminal cavity is contracted or closed. Thus, a rise in the internal pressure of the luminal cavity can be effectively restrained. Besides, since the internal volume of the luminal cavity is reduced, the influences of the luminal cavity on the surrounding tissues can be reduced.

The above-mentioned luminal cavity closing device may further include an expansion-releasing section capable of changing from a first state of being present between the arms so as to enlarge the interval of the arms against elastic forces to a second state for releasing forced expansion of the arms.

This configuration ensures that when the expansion-releasing section is brought into the second state, the interval of the arms can be instantaneously reduced, whereby the luminal cavity can be rapidly and assuredly pressed through the hollow structures adjacent to and on both sides of the luminal cavity. In addition, such an expansion-releasing section can be configured in a compact form at the distal portion of the luminal cavity closing device. Therefore, the luminal cavity closing device can be made small in diameter, which contributes to enhanced passability of the device in a blood vessel or the like.

In the above-mentioned luminal cavity closing device, a configuration may be adopted wherein the arms are each bent or curved at least at a longitudinal-directionally intermediate part thereof. The clamping mechanism can include a base part connected to the shaft, and a shape-restraining section which is provided at the base part and through which the arms are inserted. When the shape-restraining section is in a first position on the arms, the clamping mechanism assumes the expanded state, whereas when the shape-restraining section is in a second position on a distal side of the second position, the shape-restraining section deforms the arms to cause the clamping mechanism to assume the contracted state.

This configuration ensures that, by simply pushing the luminal cavity closing device toward the luminal cavity while inserting the arms in the hollow structures adjacent to and on both sides of the luminal cavity, it is possible to reduce the arm interval under the action of the shape-restraining section. This offers excellent operability (maneuverability), and makes it possible to rapidly and easily press the luminal cavity through the hollow structures adjacent to and one both sides of the luminal cavity.

The above-mentioned luminal cavity closing device may have a configuration wherein a plurality of shafts are provided which can each be rotated about an axis thereof. The arms can be curved and are provided at respective distal ends of the shafts. Orientations of the arms are changed attendantly on rotation of the shafts, whereby the clamping mechanism is caused to assume the contracted state.

This configuration enables the arm interval to be reduced by simply rotating the shafts connected to the arms which are curved. In addition, it is unnecessary to provide any complicated actuating mechanism at the distal portion of the luminal cavity closing device. Therefore, the luminal cavity closing device can be made small in diameter, which contributes to enhancement of passability of the device in a blood vessel and the like.

In the above-mentioned luminal cavity closing device, a configuration may be adopted wherein the detachment mechanism includes a bundling member through which the shafts are slidably inserted, and a heater for melting the bundling member. When the bundling member is melted under heating by the heater, the shafts are melted together with the bundling member in the melting area, whereby the clamping mechanism is detached from the shaft.

According to this configuration, the arms are not separated even after the detachment mechanism is detached from the shaft. Consequently, the state wherein the luminal cavity is pressed by the arms can be securely and stably maintained.

In the above-mentioned luminal cavity closing device, a support element capable of being inserted into the luminal cavity can be provided between the arms.

This configuration ensures that even when the arms are considerably thin (small in diameter), the luminal cavity can be pressed from both sides, since the luminal cavity is clamped between the support element and the arms. In addition, the engagement of the support element with the luminal cavity makes it possible to restrain or prevent the clamping device from being disengaged from the luminal cavity, during or after the time that the clamping mechanism is detached from the shaft.

According to another embodiment of the presently disclosed subject matter, there is provided a luminal cavity closing method that can include the following: an access step in which a luminal cavity including a flexible linear shaft, a clamping mechanism provided at a distal end of the shaft and having at least two arms, and a detachment mechanism configured to enable detachment of the clamping mechanism from the shafts is passed through a catheter inserted in a hollow lumen and advanced to a position where a luminal cavity formed in a hollow structure bifurcation area is present; an insertion step in which the arms are inserted respectively into hollow structures located adjacent to and on both sides of the luminal cavity; a pressing step in which the interval of the arms is reduced under an action of the clamping mechanism so as to press the luminal cavity through the blood vessels located adjacent to and on both sides of the luminal cavity; and a detachment step in which the clamping mechanism in the state of pressing the luminal cavity is detached from the shaft.

According to this luminal cavity closing method, a rise in the internal pressure of the luminal cavity can be effectively restrained by contracting or closing the opening (aperture) of the luminal cavity. In addition, since the internal volume of the luminal cavity is reduced, influences of the luminal cavity on the surrounding tissues can be reduced.

In the above-mentioned luminal cavity closing method, during the insertion step, the support element provided between the arms can be inserted into the luminal cavity. This ensures that even where the arms are considerably thin (small in diameter), the luminal cavity can be securely pressed from both sides, since the luminal cavity is clamped between the support element and the arms. With the support element inserted in the luminal cavity, the clamping mechanism can be restrained or prevented from being disengaged from the luminal cavity.

Thus, according to the luminal cavity closing device and the luminal cavity closing method pertaining to the presently disclosed subject matter, a rise in the internal pressure of a luminal cavity can be effectively restrained and influences of the luminal cavity on the surrounding tissues can be reduced.

### BRIEF DESCRIPTION OF THE DRAWINGS

FIG. 1 is a partial schematic side view of a luminal cavity closing device according to an embodiment of the presently disclosed subject matter;

FIG. 2 is a perspective view of a clamping mechanism of the luminal cavity closing device shown in FIG. 1;

FIG. 3A is a side sectional view showing a state wherein the clamping mechanism of the luminal cavity closing device of FIG. 1 is located in a distal portion of a catheter, FIG. 3B is a side sectional view showing a state wherein the clamping mechanism of the luminal cavity closing device of FIG. 1 has been pushed out via the distal end of an outer tube, and FIG. 3C is a side sectional view showing a state wherein the clamping mechanism of the luminal cavity closing device of FIG. 1 is closed;

FIG. 4A is a configuration view of a detachment mechanism according to a first configuration example, FIG. 4B is a configuration view of the detachment mechanism according to a second configuration example, FIG. 4C is a configuration view of the detachment mechanism according to a third configuration example, FIG. 4D is a configuration view of the detachment mechanism according to a fourth configuration example, and FIG. 4E is a configuration view of the detachment mechanism according to a fifth configuration example;

FIG. 5A is a first view illustrating a method of using the luminal cavity closing device of FIG. 1, and FIG. 5B is a second view illustrating the method of using the luminal cavity closing device of FIG. 1;

FIG. 6A is a third view illustrating a method of using the luminal cavity closing device of FIG. 1, and FIG. 6B is a fourth view illustrating a method of using the luminal cavity closing device of FIG. 1;

FIG. 7 is a fifth view illustrating a method of using the luminal cavity closing device of FIG. 1;

FIG. 8 is a perspective view showing a distal portion of a luminal cavity closing device according to another embodiment of the presently disclosed subject matter;

FIG. 9A is a side sectional view showing a state wherein a clamping mechanism of the luminal cavity closing device of FIG. 8 is expanded, and FIG. 9B is a side sectional view showing a state wherein the clamping mechanism of the luminal cavity closing device of FIG. 8 is contracted;

FIG. 10A is a first view illustrating a method of using the closing device of FIG. 8, and FIG. 10B is a second view illustrating a method of using the luminal cavity closing device of FIG. 8;

FIG. 11A is a third view illustrating a method of using the luminal cavity closing device of FIG. 8, and FIG. 11B is a fourth view illustrating a method of using the luminal cavity closing device of FIG. 8;

FIG. 12 is a fifth view illustrating a method of using the luminal cavity closing device of FIG. 8;

FIG. 13A is a side sectional view showing a distal portion of a luminal cavity closing device according to another embodiment of the presently disclosed subject matter, and FIG. 13B is a side sectional view showing a state wherein a clamping mechanism of the luminal cavity closing device of FIG. 13A is pushed out via the distal end of an outer tube;

FIG. 14A is a first view illustrating a method of using the luminal cavity closing device of FIG. 13A, and FIG. 14B is a second view illustrating a method of using the luminal cavity closing device of FIG. 13A;

FIG. 15A is a third view illustrating a method of using the luminal cavity closing device of FIG. 13A, and FIG. 15B is a fourth view illustrating a method of using the luminal cavity closing device of FIG. 13A; and

FIG. 16 is a fifth view illustrating a method of using the luminal cavity closing device of FIG. 13A.

### DETAILED DESCRIPTION OF EXEMPLARY EMBODIMENTS

Now, luminal cavity closing devices and luminal cavity closing methods in accordance with principles of the presently disclosed subject matter will be described below, with respect to exemplary embodiments and referring to the accompanying drawings.

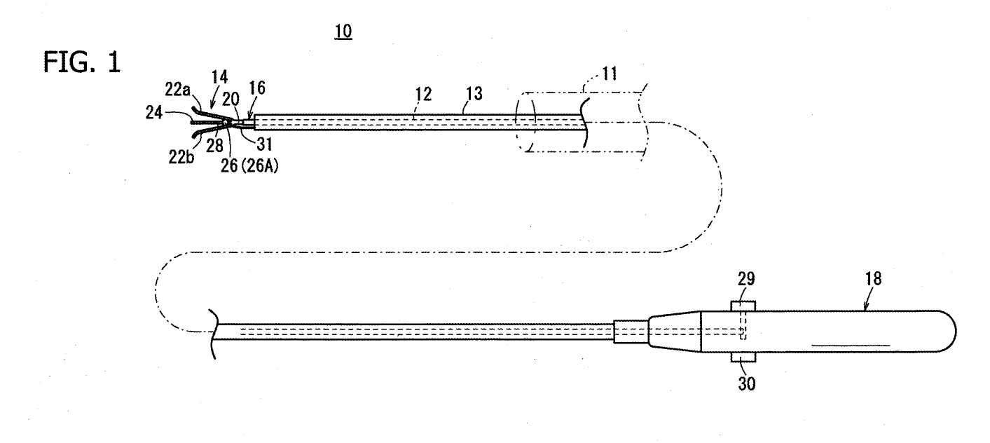

FIG. 1 is a partial schematic side view showing the configuration of a luminal cavity closing device 10 according to an embodiment of the presently disclosed subject matter. The luminal cavity closing device 10 includes a long outer tube 13 which can be inserted in a catheter 11, a long shaft 12 inserted and disposed in the outer tube 13, a clamping mechanism 14 connected to the shaft 12, and a detachment mechanism 16 configured to enable detachment of the clamping mechanism 14 from the shaft 12. The luminal cavity closing device 10 is a device that can be used, for example, in a therapeutic procedure wherein a luminal cavity generated in a blood vessel bifurcation area is pressed through blood vessels on both sides to reduce the internal volume of the luminal cavity, after which the clamping mechanism 14 in the state of pressing the luminal cavity is detached from the shaft 12, whereby the blood pressure inside the luminal cavity is lowered so as to prevent rupture of the luminal cavity. Now, the configuration of the components of the device will be described below.

As shown in FIG. 1, the outer tube 13 is a hollow tubular member which is flexible, long, linear (wire-like), and open at both ends thereof. At a proximal portion of the outer tube 13, a grip section 18 is provided which is larger in diameter than the shaft 12 and is to be gripped by the operator. The outer tube 13 can be made with a degree of flexibility such that it can easily follow up and through the curving portions of a living body lumen (blood vessel or the like), together with the catheter 11, when inserted into and fed through the lumen.

Examples of the material for forming the outer tube 13 include metals and resins. Examples of the metals include pseudo-elastic alloys (inclusive of superelastic alloys) such as Ni—Ti alloys, shape memory alloys, stainless steels (e.g., all types of SUS, such as SUS304, SUS303, SUS316, SUS316L, SUS316J1, SUS316J1L, SUS405, SUS430, SUS434, SUS444, SUS429, SUS430F, SUS302, etc.), cobalt alloys, noble metals such as gold, platinum, etc., tungsten alloys, and carbon-containing materials (inclusive of piano wire). Examples of the resins include polymer materials such as polyolefins (e.g., polyethylene, polypropylene, polybutene, ethylene-propylene copolymers, ethylene-vinyl acetate copolymers, ionomers, or mixtures thereof), polyvinyl chloride, polyamides, polyamide elastomers, polyesters, polyester elastomers, polyurethane, polyurethane elastomers, polyimides, fluoro-resins, and mixtures of them, which may be used either singly or in combination of two or more of them. The outer tube 13 may be composed of a multi-layer tube or the like of a composite material formed from these metals and/or resins.

The size of the outer tube 13 is appropriately selected according to the target part to be treated. For instance, in the case where the luminal cavity closing device is to be used for therapy of cerebral aneurysm, the overall length of the outer tube 13 can be about 800 to 1,500 mm, and the outside diameter can be about 0.6 to 3 mm. In addition, depending on the target part to be treated, the overall length of the outer tube 13 can be about 300 to 2,000 mm, the outside diameter can be about 5 to 10 mm, and the inside diameter about 4 to 9.8 mm.

The shaft 12 is inserted and disposed in the lumen of the outer tube 13 so as to be displaceable in the axial direction. The proximal end of the shaft 12 is connected to an operating knob 29 provided at the grip section 18. With the operating knob 29 operated in the axial direction, the shaft 12 can be advanced and retracted relative to the outer tube 13 and can be rotated relative to the outer tube 13.

The shaft 12, like the outer tube 13, can have a degree of flexibility such that it can easily follow up and through curving portions of a living body lumen (blood vessel or the like) when inserted into and fed through the lumen. In addition, the shaft 12 can have an appropriate degree of rigidity such that it can push out the clamping mechanism 14 from the outer tube 13 and can transmit torque to the clamping mechanism 14. In view of this, the shaft 12 can be formed from any of the materials mentioned above as material for the outer tube 13.

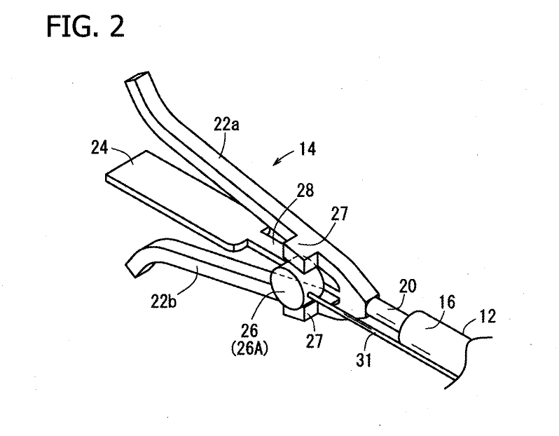

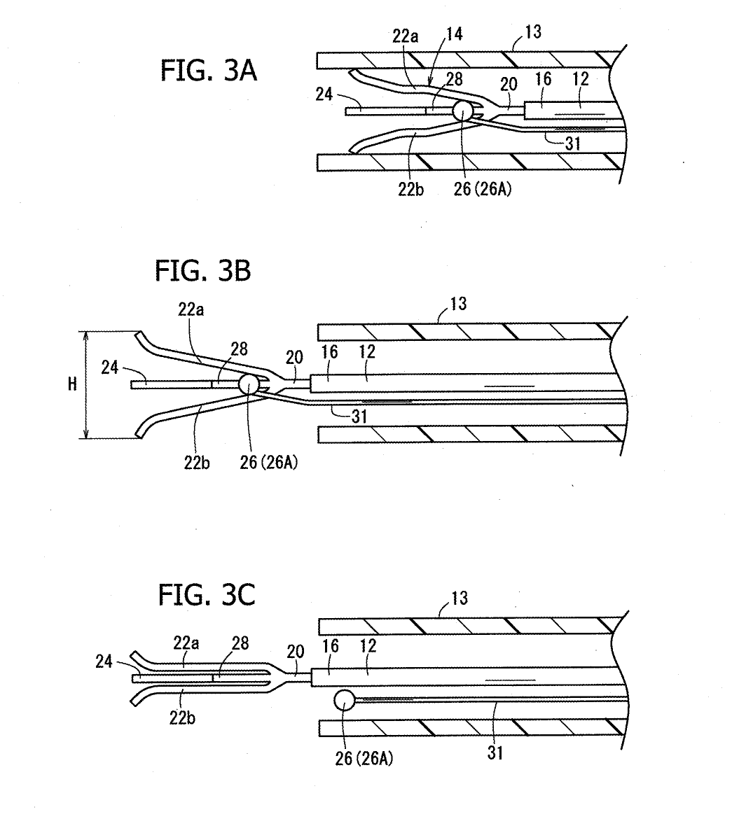

As shown in FIG. 2, the clamping mechanism 14 in this exemplary embodiment includes: a base part 20 connected to the shaft 12; at least plural (in the example shown, two) arms 22a and 22b branched from a distal-side portion of the base part 20; a support element 24 disposed between the arms 22a and 22b; and an expansion-releasing section 26 disposed between the arms 22a and 22b. The clamping mechanism 14 can operate from an expanded state (see FIG. 3B) wherein the interval of the arms 22a and 22b is enlarged to a contracted state (see FIG. 3C) wherein the interval of the arms 22a and 22b is reduced.

The arms 22a and 22b can be formed integrally with the base part 20, and are so configured that they can be inserted into blood vessels adjacent to and on both side of a bosselation (or other abnormality or injury) present in a blood vessel bifurcation area. Specifically, base ends (proximal ends) of the arms 22a and 22b are connected to the base part 20, and the arms 22a and 22b are branched from the base part 20 and extended along the distal direction. Distal portions of the arms 22a and 22b are bent in outward directions (in the directions in which the arms 22a and 22b are spaced away from each other).

The support element 24 disposed between the arms 22a and 22b is provided on an extension line of the base part 20, and has a flat plate-like shape greater in width than the arms 22a and 22b. Between the support element 24 and the base part 20, a link section 28 is formed which extends from the base part 20 between the arms 22a and 22b. The support element 24 is supported by the link section 28. The link section 28 is smaller in width than the support element 24. The expansion-releasing section 26 is disposed at a position adjacent to the link section 28.

The expansion-releasing section 26 is so configured that it can change from a first state of being present between the arms 22a and 22b so as to enlarge the interval of the arms 22a and 22b to a second state for releasing the forced expansion of the arms 22a and 22b. In this embodiment, specifically, the expansion-releasing section 26 is configured as an intermediate member 26A which is held in the state of being clamped between the arms 22a and 22b on the proximal side of the arms 22a and 22b.

The arms 22a and 22b are forcibly elastically deformed by the intermediate member 26A, and the intermediate member 26A is held in a state such that the arms 22a and 22b are opened wider than in a natural state. Specifically, in the state wherein the intermediate member 26A is clamped between the arms 22a and 22b, as shown in FIG. 2, the interval of (or distance between) the arms 22a and 22b is gradually enlarged toward the distal side. Thus, the two arms 22a and 22b are in a V-shaped form with a certain angle therebetween. In the case where the arms 22a and 22b are provided with bulging parts 27 on the proximal side and the intermediate member 26A is clamped between the bulging parts 27 as in the example shown, the intermediate member 26A can be assuredly clamped between the arms 22a and 22b even in the presence of the link section 28 for providing the support element 24.

As shown in FIG. 3A, the arms 22a and 22b with the expansion-releasing member 26 clamped therebetween may be stored in the outer tube 13 in a state wherein expansion thereof is restrained by the inner circumferential surface of the outer tube 13 (a state wherein their portions on the distal side of the position of clamping the intermediate member 26A are elastically deformed). In this state, the arms 22a and 22b are receiving forces acting in directions in which the arms 22a and 22b are forcibly expanded away from each other by the intermediate member 26A clamped between them. Therefore, when the arms 22a and 22b are protruded from the distal end of the outer tube 13, as shown in FIG. 3B, those portions of the arms 22a and 22b which are located on the distal side of the position of contact with the intermediate member 26A are elastically restored into their original shapes. This results in a state in which the arms 22a and 22b are spaced wider from each other (the interval between them is enlarged).

A wire 31 is connected to the intermediate member 26A. The wire is inserted in the outer tube 13, and is connected on the proximal side to an operating knob 30 (releasing operating section) provided at the grip section 18. When the operating knob 30 is operated and the wire 31 is pulled in the proximal direction, the intermediate member 26A is released from the position between the arms 22a and 22b, as shown in FIG. 3C. As a result, the arms 22a and 22b are each displaced in a direction in which they come closer to each other by their elastic restoring forces. Consequently, the clamping mechanism 14 is put into the contracted state wherein the interval of the arms 22a and 22b has been reduced. A radiopaque marker may be provided on the whole body of the clamping mechanism 14 or on the arms 22a and 22b, so as to enable visible checking under radioscopy.

Examples of the materials for forming the base part 20, the arms 22a, 22b and the support element 24 include elastic or superelastic metallic materials such as stainless steels, tantalum, cobalt alloys, titanium alloys, Ni—Ti alloys, etc., and various polymers such as polyolefins such as polyethylene, polypropylene, ethylene-vinyl acetate copolymer, etc., polyvinyl chloride, polymethyl methacrylate, polycarbonates, polybutadiene, polyamides, polyesters, etc.

The dimensions of the clamping mechanism 14 in the state in which it is protruded from the distal end of the outer tube 13 and the expansion-releasing section 26 is disposed between the arms 22a and 22b (the state shown in FIG. 2) are appropriately selected according to the target part to be treated. For example, in the case where the luminal cavity closing device is used for treatment of cerebral aneurysm, the overall length of the clamping mechanism 14 can be about 2 to 50 mm, and the width H between the distal ends of the arms 22a and 22b in the expanded state (see FIG. 3B) can be about 2 to 30 mm.

Now, the detachment mechanism 16 shown in FIG. 1 will be described below. The detachment mechanism 16 is so configured as to be able to disconnect the clamping mechanism 14 from the shaft 12 and be able to transmit torque to the clamping mechanism 14 side in the condition where the shaft 12 and the detachment mechanism 16 are connected to each other. The detachment mechanism 16 having such a function can be formed by adopting a configuration wherein two members composed separately are disconnectably connected by physical engagement (fitting, hooking, or the like) or a configuration wherein two members are connected so as to be disconnectable by splitting a member by some physical action (thermal action, chemical action or the like). While some configuration examples of the detachment mechanism 16 will be described below, the detachment mechanism 16 naturally is not restricted to these configuration examples.

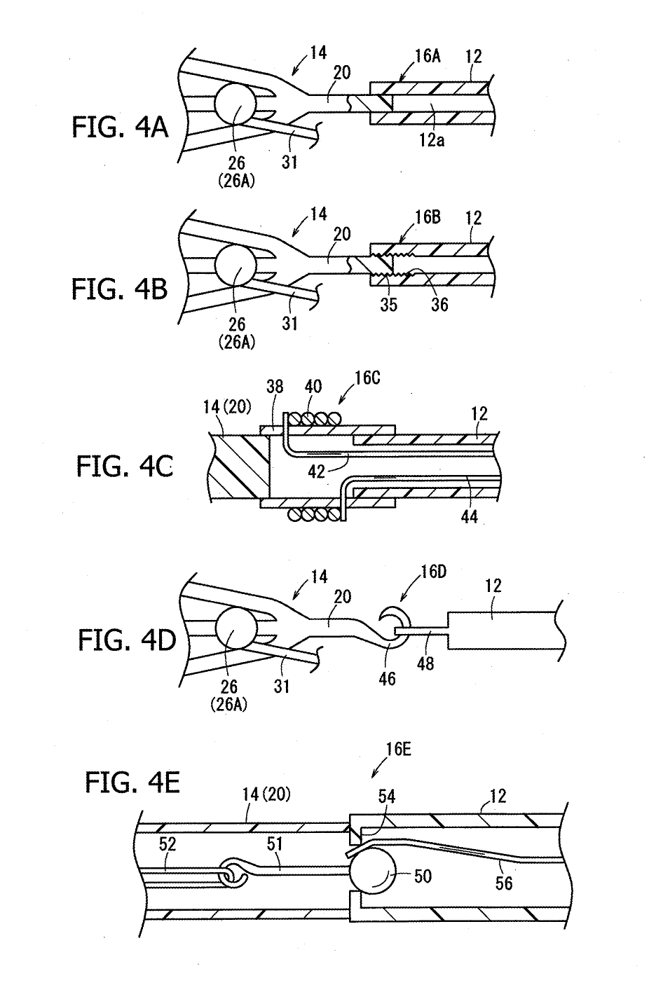

A detachment mechanism 16A according to a first configuration example shown in FIG. 4A has a configuration in which the base part 20 of the clamping mechanism 14 and the shaft 12 are coupled together by fitting. The fitting force (coupling force) between the base part 20 and the shaft 12 is set so that the connected state is maintained through fitting when a force for separating the two members is below a predetermined value, but that the clamping mechanism 14 and the shaft 12 are detached from each other because the fitting is canceled when a force at or above the predetermined value is exerted. According to the detachment mechanism 16A, the clamping mechanism 14 can be automatically detached from the shaft 12 by retracting the shaft 12, after a luminal cavity is pressed by the clamping mechanism 14 through blood vessels adjacent to and on both sides of the luminal cavity. Incidentally, while the base part 20 is inserted into a fitting hole 12a provided in the shaft 12 (and both members are thereby fitted with respect to each other) in FIG. 4A, the male-female relationship in the fitting may be reversed. In other words, a configuration may be adopted wherein the base part 20 of the clamping mechanism 14 is provided with a fitting hole, and the shaft 12 is provided at its distal end with a fitting projection capable of being fitted into the fitting hole.

A detachment mechanism 16B according to a second configuration example shown in FIG. 4B is configured so that the base part 20 of the clamping mechanism 14 and the shaft 12 are connected by screw engagement. Specifically, the base part 20 is formed with a male screw 35 at an outer circumferential portion thereof, while the shaft 12 is formed with a female screw 36 at an inner circumferential portion thereof. The male screw 35 and the female screw 36 are so formed that the screw engagement is released according to the direction of rotation of the shaft 12 at the time of the procedure for closing a luminal cavity. The screw engagement force (coupling force) between the base part 20 and the shaft 12 is so set that the screw engagement is maintained when the torque acting between them is below a predetermined value, but that the screw engagement is released to and the clamping mechanism 14 and the shaft 12 can be thereby detached from each other when a torque at or above the predetermined value is exerted.

According to the detachment mechanism 16B, when the torque exerted by rotating the shaft 12 reaches or exceeds a predetermined value after a luminal cavity is pressed by the clamping mechanism 14 through blood vessels adjacent to and on both sides of the luminal cavity, the screw engagement between the clamping mechanism 14 and the shaft 12 is canceled (or disengaged). As a result, the clamping mechanism 14 can be automatically detached from the shaft 12. Incidentally, while a male screw 35 provided on the base part 20 is shown as engaged with a female screw 36 provided on the shaft 12 to achieve connection between both the members in FIG. 4B, the male-female relationship in the screw engagement may be reversed. In other words, a configuration may be adopted wherein the clamping mechanism 14 is provided with a female screw at the base part 20 thereof, while the shaft 12 is provided with a male screw at the distal end thereof.

A detachment mechanism 16C according to a third configuration example shown in FIG. 4C includes: a link section 38 by which the base part 20 of the clamping mechanism 14 and the distal end of the shaft 20 are linked to each other; a heater 40 wound in a coil form around the outer circumference of the link section 38; and first and second lead wires 42 and 44 connected to the heater 40. The link section 38 is formed from a material capable of rupture by melting when heated (e.g., resin, metal having a low melting-point, or the like). The first lead wire 42 is connected to one end of the heater 40, while the second lead wire 44 is connected to the other end of the heater 40. The first and second lead wires 42 and 44 are inserted in the shaft 12, and are connected to a power supply at their ends on the opposite side to the side of connection with the heater 40. The power supply may be either direct current (DC) or alternate current (AC).

The detachment mechanism 16C ensures that after a luminal cavity is pressed by the clamping mechanism 14 through blood vessels adjacent to and on both sides of the luminal cavity, the clamping mechanism 14 can be detached from the shaft 12 by energizing the heater 40 through the first and second lead wires 42 and 44 to generate heat, thereby causing rupture (fracture by fusing) of the link section 38.

A detachment mechanism 16D according to a fourth configuration example shown in FIG. 4D includes a hook section 46 provided at the base part 20 of the clamping mechanism 14, and an engagement section 48 provided at the distal end of the shaft 12 for engagement with the hook section 46. The engaging force (coupling force) between the hook section 46 and the engagement section 48 is so set that when the shaft 12 is pushed out slightly toward the distal side in the condition where a luminal cavity is pressed by the clamping mechanism 14 through blood vessels adjacent to and on both sides of the luminal cavity, the engagement between the hook section 46 and the engagement section 48 is released and, therefore, the clamping mechanism 14 can be detached from the shaft 12. According to the detachment mechanism 16D, when the shaft 12 is rotated in the condition where the luminal cavity is pressed through the blood vessel adjacent to and on both sides of the luminal cavity, the hook engagement between the clamping mechanism 14 and the shaft 12 is canceled (i.e., is disengaged), so that the clamping mechanism 14 can be automatically detached from the shaft 12.

A detachment mechanism 16E according to a fifth configuration example shown in FIG. 4E includes: an intermediate member 51 having a ball-shaped part 50 at the proximal end thereof; a pulling member 52 for pulling the intermediate member 51 toward the inside of the base part 20 of the clamping mechanism 14; a holding ring 54 provided at a distal portion of the shaft 12; and a release wire 56 inserted in the shaft 12. The release wire 56 is, for example, connected to an operating part (slide knob or the like) provided at the grip section 18, and is pulled back in the proximal direction by an operation on the operating section.

In the state where the release wire 56 is clamped between the holding ring 54 and the ball-shaped part 50 (the state wherein the ball-shaped part 50 and the release wire 56 are immobilized), as shown in FIG. 4E, the ball-shaped part 50 is inhibited from moving into the base part 20 through the inside of the holding ring 54. Therefore, a connected state between the clamping mechanism 14 and the shaft 12 is maintained. On the other hand, when the operating section is operated to pull the release wire 56 in the proximal direction, the release wire 56 is caused to be removed from between the holding ring 54 and the ball-shaped part 50. This action results in the ball-shaped part 50 being able to pass through the inside of the holding ring 54. Therefore, the ball-shaped part 50 is moved into the base part 20 under the pulling action of a pulling member 52. Consequently, the clamping mechanism 14 can be detached from the shaft 12.

Other configurations which can be adopted for the detachment mechanism 16 include a configuration wherein the base part 20 of the clamping mechanism 14 and the distal end of the shaft 12 are linked to each other by a metallic link section, and detachment of the clamping mechanism 14 from the shaft 12 is effected through electrolysis of the link section. Also included is a configuration wherein a link section between the base part 20 of the clamping mechanism 14 and the shaft 12 is cut off from the shaft 12 by applying a fluid pressure to the connection part. The fluid pressure can cause a mechanical change (structural change) or chemical reaction to occur in order to achieve the detachment. For example, a water soluble material link can be formed between the clamping mechanism 14 and shaft 12 such that the link dissolves under fluid pressure. Alternatively, fluid pressure can move a first structure relative to a second structure to unlock the two structures from each other.

The luminal cavity closing device 10 according to this embodiment can be fundamentally configured as described above. Now, the operation and effect of the luminal cavity closing device 10 will be described below, in relation to an exemplary method of using the luminal cavity closing device 10 (luminal cavity closing method). The luminal cavity closing method using the luminal cavity closing device 10 can include the following steps, which can be executed chronologically or in any order deemed appropriate by the practitioner.

(1) Access Step (First Step)

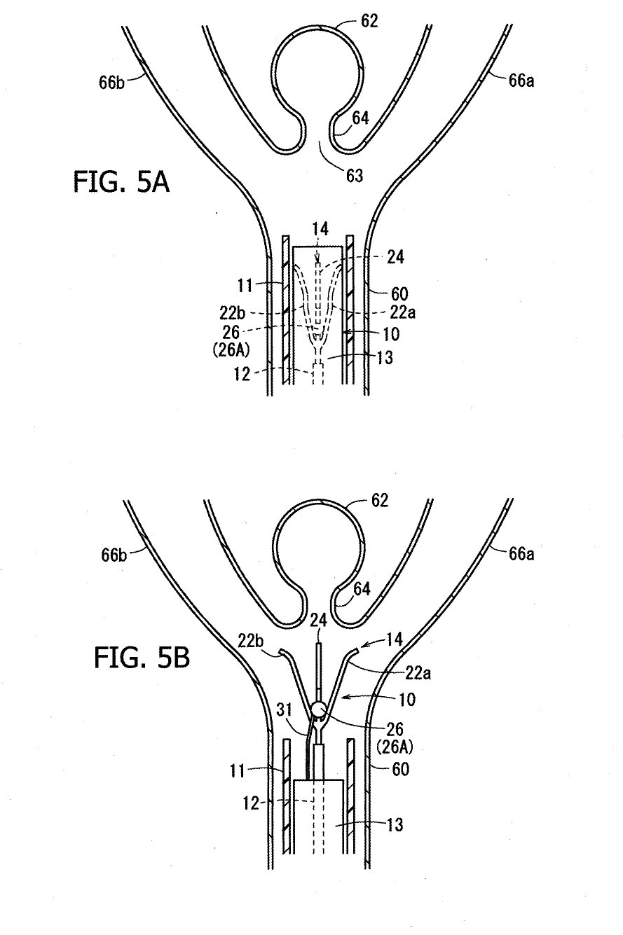

In an access step, as shown in FIGS. 5A and 5B, the luminal cavity closing device 10 is passed through the catheter 11 and disposed in the vicinity of a luminal cavity (e.g., aneurysm or varicose portion) 62 which is the target part to be treated (e.g., closed). The luminal cavity 62 which is the object of therapy in this example is a hollow sac-like tissue generated in a lumen bifurcation area (blood vessel bifurcation area) in a living body. While the blood vessel shown in the drawings is bifurcated in a Y-shaped form, it may be branched in a T-shaped or other form. The luminal cavity 62 is composed of a part whose outer surface is protuberant in such a manner that the lumen tissue is partly swollen outward. The protuberant part has an inside space, which communicates with the lumen through an opening 63. In the luminal cavity 62, the part protruding to the outside forms a sac-like structure, of which the inside space is separated from the outside by a membrane-like structure formed by part of the tissue. The luminal cavity 62 may in some cases have the opening 63 (communicating with the lumen) being narrowed to form a neck part 64, or may in other cases have the opening 63 being comparatively large so as not to form a neck part. Examples of the blood vessel where the luminal cavity 62 is generated include arteries, veins, and peripheral vessels. Examples of the luminal cavity 62 include cerebral aneurysm, abdominal artery aneurysm, thoratic artery aneurysm, coronary artery aneurysm, popliteal artery aneurysm, femoral artery aneurysm, and carotid artery aneurysm.

In this access step, specifically, first, the catheter 11 with a guide wire inserted therein is fed through the blood vessel 60, and the distal end of the catheter 11 is caused to reach a blood vessel bifurcation area or the vicinity thereof. Thereafter, the guide wire is pulled out of the catheter 11. After the guide wire is pulled out of the catheter 11, the luminal cavity closing device 10 with the clamping mechanism 14 stored in the outer tube 13 is inserted into the catheter 11. In this case, as shown in FIG. 5A, the distal end of the outer tube 13 of the closing device is located at a position slightly spaced from the luminal cavity 62. Next, while maintaining the positions of the catheter 11 and the outer tube 13, the operating knob 29 (see FIG. 1) is operated to advance the shaft 12 in the distal direction. By this operation, as shown in FIG. 5B, the clamping mechanism 14 is caused to protrude from the outer tube 13, so as to be disposed in the blood vessel bifurcation area. In this instance, the clamping mechanism 14 expands into the expanded state, since the expansion-releasing section 26 is located between the arms 22a and 22b of the clamping mechanism 14.

(2) Insertion Step (Second Step)

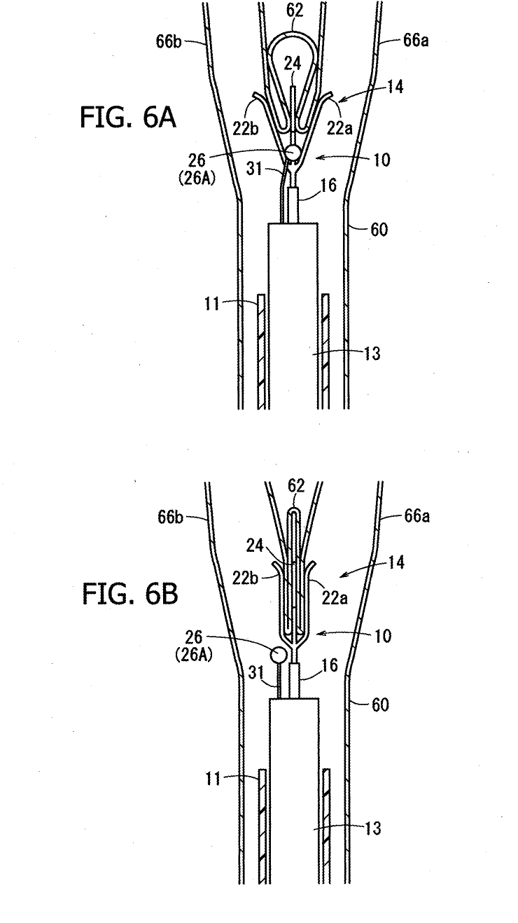

Subsequently, as shown in FIG. 6A, while holding the position of the catheter 11, the shaft 12 is advanced in the distal direction together with the outer tube 13. By this operation, the arms 22a and 22b of the clamping mechanism 14 are inserted respectively into blood vessels 66a and 66b adjacent to and on both sides of the luminal cavity 62. In this insertion step, the blood vessels 66a and 66b adjacent to and on both sides of the bosselation or other abnormality or extension of the luminal cavity 62 are pressed toward the inner side (toward the luminal cavity 62) by the arms 22a and 22b thus inserted. In addition, the luminal cavity 62 present between the blood vessels 66a and 66b is also pressed. As a result, the internal volume of the luminal cavity 62 is reduced to some extent. In this embodiment, the support element 24 is provided between the arms 22a and 22b. Attendant on the insertion of the arms 22a and 22b into the blood vessels, therefore, the support element 24 is inserted through the opening 63 into the luminal cavity 62.

(3) Pressing Step (Third Step)

Next, as shown in FIG. 6B, the arms 22a and 22b are closed under the action of the clamping mechanism 14, whereby the luminal cavity 62 is further pressed through the blood vessels 66a and 66b adjacent to and on both sides of the luminal cavity 62. Specifically, the wire 31 is pulled in the proximal direction, whereby the intermediate member 26A is released from between the arms 22a and 22b. In this instance, the arms 22a and 22b are displaced in directions so as to reduce their interval by their elastic restoring forces, thereby pressing and contracting the luminal cavity 62 through the blood vessels 66a and 66b adjacent to and on both sides of the luminal cavity 62. As a result, the opening 63 of the luminal cavity 62 is contracted or closed, and the internal volume of the luminal cavity 62 is remarkably reduced as compared with the volume of the cavity 62 before the procedure (the state of FIG. 5A).

The support element 24 acts to press and contract the luminal cavity 62. Specifically, when the arms 22a and 22b are displaced inward by their elastic restoring forces, the luminal cavity 62 is clamped between the support element 24 and the arm 22a on one side, and the luminal cavity 62 is clamped between the support element 24 and the arm 22b on the other side. Therefore, even in the case where the positions of the arms 22a and 22b are out of register and the luminal cavity 62 cannot be clamped between the arms 22a and 22b, it is possible to press and contract the luminal cavity 62 from both sides in a reliable manner. While the support element 24 is clamped between the arms 22a and 22b through the luminal cavity 62 therebetween, the support element 24 may be provided with rugged patterns on its surfaces to which the arms 22a and 22b can fit.

(4) Detachment Step (Fourth Step)

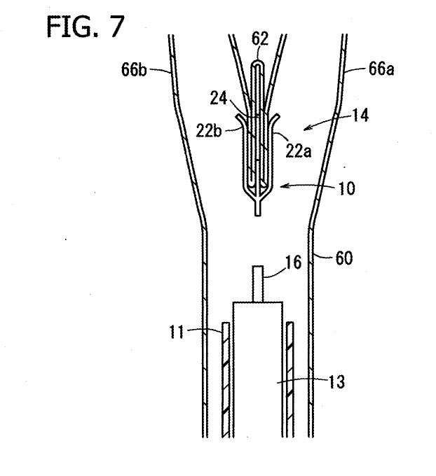

Subsequently, as shown in FIG. 7, the shaft 12 and the clamping mechanism 14 can be detached from each other under an action of the detachment mechanism 16. The operation for detaching the clamping mechanism 14 from the shaft 12 depends on the configuration of the detachment mechanism 16.

In the case of the detachment mechanism 16A shown in FIG. 4A, when a force in excess of the fitting force between the distal end of the shaft 12 and the base part 20 of the clamping mechanism 14 is exerted via the rotation of the shaft 12, and when the luminal cavity 62 is pressed by the clamping mechanism 14 through the blood vessels adjacent to and on both sides of the luminal cavity 62, the fitting is released. As a result, the clamping mechanism 14 can be detached from the shaft 12. Or, alternatively, the fitting between the distal end of the shaft 12 and the base part 20 of the clamping mechanism 14 may be canceled (or disengaged) by retracting the shaft 12. In the case of the detachment mechanism 16B shown in FIG. 4B, when the shaft 12 is rotated while the luminal cavity 62 is pressed by the clamping mechanism 14 through the blood vessels adjacent to and on both sides of the luminal cavity 62, the screw engagement between the distal end of the shaft 12 and the base part 20 of the clamping mechanism 14 is released. Consequently, the clamping mechanism 14 can be detached from the shaft 12.

In the case of the detachment mechanism 16C shown in FIG. 4C, the clamping mechanism 14 can be detached from the shaft 12 by energizing the heater 40 to thereby cause rupture (by melting) of the link section 38. In the case of the detachment mechanism 16D shown in FIG. 4D, when the shaft 12 is slightly pushed back toward the distal side or rotated while the luminal cavity 62 is pressed by the clamping mechanism 14 through the blood vessels adjacent to and on both sides of the luminal cavity 62, the engagement between the hook section 46 provided at the base part 20 of the clamping mechanism 14 and the engagement section 48 provided at the distal end of the shaft 12 is released. As a result, the clamping mechanism 14 can be detached from the shaft 12. In the case of the detachment mechanism 16E shown in FIG. 4E, the clamping mechanism 14 can be detached from the shaft 12 by operating the operating part provided at the grip section 18 so as to pull out the release wire 56 from between the ball-shaped part 50 and the holding ring 54 (so as to release the release wire 56 from the coupling with the ball-shaped part 50) and to move the ball-shaped part 50 into the base part 20 (to pull off the release wire 56 toward the proximal side).

As described above, and according to methods for using the luminal cavity closing device 10 in this embodiment, it is possible to carry out a procedure (luminal cavity closing method) wherein the luminal cavity closing device 10 is inserted into a catheter, the clamping device 14 of the luminal cavity closing device 10 is delivered into the vicinity of a luminal cavity 62 generated in a blood vessel bifurcation area, the luminal cavity 62 is pressed by the arms 22a and 22b of the clamping mechanism 14 through the blood vessels 66a and 66b adjacent to and on both sides of the luminal cavity 62, and thereafter the clamping mechanism 14 in the state of pressing the luminal cavity 62 is detached from the shaft 12.

When the luminal cavity 62 is pressed through the blood vessels 66a and 66b located on both sides of the luminal cavity 62, the opening 63 of the luminal cavity 62 is contracted or closed, so that a rise in the internal pressure of the luminal cavity 62 can be effectively restrained and/or reduced. Specifically, the internal pressure of the luminal cavity 62 can be lowered, since it becomes insusceptible (or substantially insusceptible) to the influence of the pressure of the blood flowing through the blood vessel (parent vessel). Particularly, where the opening 63 of the luminal cavity 62 is closed, the inside of the blood vessel and the inside of the luminal cavity 62 are shut off from each other, which is highly effective in restraining or preventing the internal pressure of the luminal cavity 62 from rising. Consequently, the danger of rupture of the luminal cavity 62 can be effectively reduced and/or eliminated.

In addition, since the internal volume of the luminal cavity 62 is reduced, the internal pressure of the luminal cavity 62 can be restrained or prevented from rising, and the influence of the luminal cavity 62 on the surrounding tissues can be reduced. Specifically, for example, in the case of cerebral aneurysm, a reduction in size of the cerebral aneurysm makes it possible to lighten any pressing on the cerebral tissues surrounding the cerebral aneurysm, and to further enhance the therapeutic effect.

As mentioned above, in this embodiment, the expansion-releasing section 26 can be provided which can change from a first state in which the expansion-releasing section 26 is present between the arms 22a and 22b so as work against elastic forces to enlarge the interval between the arms 22a and 22b, to the second state in which the expansion of the arms 22a and 22b is released. Therefore, when the expansion-releasing section 26 is in the second state, the interval of the arms 22a and 22b can be instantly reduced, whereby the luminal cavity 62 can be rapidly and assuredly pressed through the blood vessels 66a and 66b adjacent to and on both sides of the luminal cavity 62. The expansion-releasing section 26 can be configured compactly at the distal portion of the luminal cavity closing device 10. Therefore, the luminal cavity closing device 10 can be made small in diameter, which contributes to enhancement of passability of the device in a lumen.

In this embodiment, the expansion-releasing section 26 can be composed of the intermediate member 26A that is releasably clamped between the arms 22a and 22b. The expansion-releasing section 26 is so configured that when the intermediate member 26A is released from between the arms 22a and 22b, the arms 22a and 22b are displaced inward by their elastic restoring forces, whereby the arms 22a and 22b are closed. Accordingly, the luminal cavity 62 can be rapidly and reliably pressed through the blood vessels 66a and 66b adjacent to and on both sides of the luminal cavity 62, while adopting a simple configuration.

Incidentally, the expansion-releasing section 26 is not restricted to the above-mentioned intermediate member 26A. In another example, the expansion-releasing section 26 may be composed of a member made of a resin or metal having a low melting-point which undergoes rupture by melting when supplied with an electric current. According to this example, the expansion-releasing section 26 is, before melting, present between the arms 22a and 22b so as to enlarge the interval of the arms 22a and 22b against their elastic forces, but, when melted, releases the forced expansion of the arms 22a and 22b. Therefore, according to this example, the luminal cavity 62 can be rapidly and assuredly pressed through the blood vessels 66a and 66b adjacent to and on both sides of the luminal cavity 62.

In this embodiment, the support element 24 which can be inserted into a luminal cavity 62 is provided between the arms 22a and 22b. Owing to a guiding action based on the insertion of the support element 24 into the luminal cavity 62, therefore, the arms 22a and 22b can be disposed in appropriate positions on both sides of the luminal cavity 62 (e.g., bosselation). In addition, even where the arms 22a and 22b are considerably thin (small in diameter), the luminal cavity 62 can be assuredly pressed from both sides, since the luminal cavity 62 is clamped between the support element 24 and the arms 22a, 22b. Furthermore, when the luminal cavity 62 is contracted by being pressed by the arms 22a and 22b, the support element 24 is hooked in the luminal cavity 62. This ensures that during and after the action of detaching the clamping mechanism 14 from the shaft 12 occurs, the clamping mechanism 14 can be restrained or prevented from being disengaged from the luminal cavity 62.

Incidentally, while an example wherein the support element 24 which is inserted into the luminal cavity 62 is left indwelling in situ has been described in this embodiment, the support element 24 may be configured to be pulled out of the luminal cavity 62 after the arms 22a and 22b are inserted into the blood vessels on both sides of the luminal cavity 62. Alternatively, the support element 24 may be omitted altogether. The point that the support element 24 may be pulled out of the luminal cavity 62, and the point that the support element 24 may be omitted, are applicable also in any of the embodiments which will be described later and for those embodiments not specifically described herein.

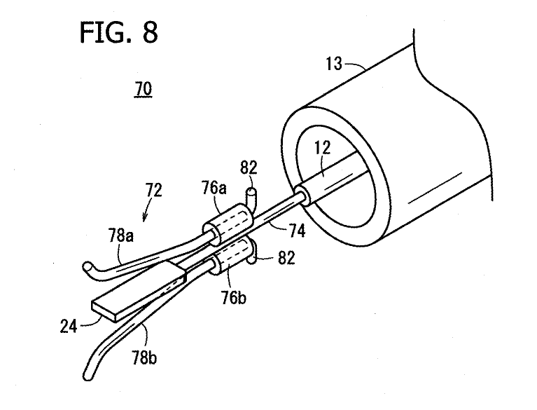

FIG. 8 is a partial schematic side view of a luminal cavity closing device 70 according to another embodiment of the presently disclosed subject matter. Incidentally, in the luminal cavity closing device 70, elements exhibiting functions and effects that are the same as or similar to those of the elements of the luminal cavity closing device 10 according to the above-described embodiment are denoted by the same reference symbols as used above, and detailed descriptions of them will be omitted.

The luminal cavity closing device 70 according to this embodiment differs from the luminal cavity closing device 10 of the first embodiment at least in the configuration of a clamping mechanism 72. The clamping mechanism 72 is a mechanism linked to a distal portion of the shaft 12. As shown in FIG. 8, the clamping mechanism 72 can include: a base part 74 linked to the shaft 12; plural (in the example shown, two) shape-restraining sections 76a and 76b provided at the base part 74; plural (in the example shown, two) arms 78a and 78b inserted respectively through the shape-restraining sections 76a and 76b; and, a support element 24 disposed between the arms 78a and 78b.

In this embodiment, the shape-restraining sections 76a and 76b are each a rectilinear tubular body with an opening at each of the ends thereof. The shape-restraining sections 76a and 76b are provided substantially in parallel (parallel or almost parallel) to the base part 74, respectively, on the sides of a one-side surface and an other-side surface of the support element 24, specifically, at positions opposite to each other with the base part 74 therebetween.

The arms 78a and 78b are so formed that in a natural state (a state in which the arms are not deformed elastically), they are rectilinear in a fixed range on the proximal side, and are bent at intermediate portions thereof so that their interval increases along the distal direction. Distal portions of the arms 78a and 78b are bent toward outer sides (away from the support elements 24). Proximal portions of the arms 78a and 78b are bent to outer sides, to constitute slip-off preventive sections 82 for preventing the arms 78a and 78b from slipping off distally from the shape-restraining sections 76a and 76b. The outside diameter of the arms 78a 78b and the inside diameter of the shape-restraining sections 76a and 76b are set to be roughly equal. Thus, the arms 78a and 78b are slidably inserted in the shape-restraining sections 76a and 76b in the condition where a certain degree of frictional resistance is present between the outer circumferential surfaces of the arms 78a and 78b and the inner circumferential surfaces of the shape-restraining sections 76a and 76b.

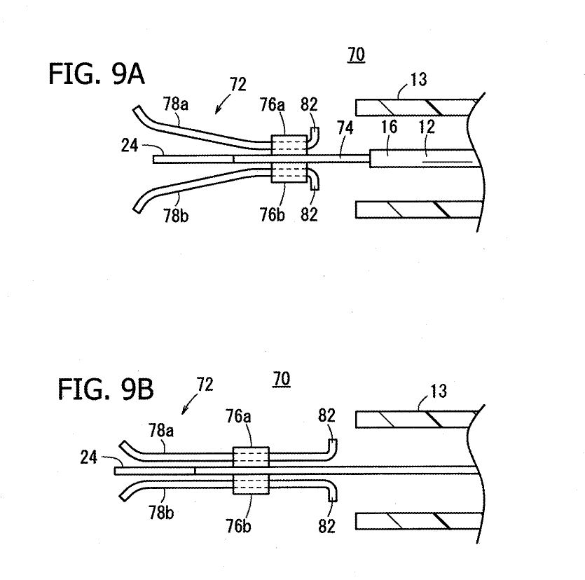

When the shape-restraining sections 76a and 76b are located at positions (first positions) on the proximal side of the arms 78a and 78b, as shown in FIG. 9A, the clamping mechanism 72 configured as described above assumes an expanded state (a state wherein the interval of the arms 78a and 78b has been enlarged). On the other hand, when the shape-restraining sections 76a and 76b are located at positions (second positions) deviated distally from the first positions, as shown in FIG. 9B, the clamping mechanism 72 assumes a contracted state (a state in which the interval of the arms 78a and 78b has been reduced).

Incidentally, as shown in FIG. 9A, the width between the distal ends of the arms 78a and 78b in the natural state may be greater than the inside diameter of the outer tube 13. In this case, the clamping mechanism 72 before protruding from the distal end of the outer tube 13 is stored in the outer tube 13 in a state (a state of being contracted by elastic deformation) in which its expansion is restrained by the inner circumferential surface of the outer tube 13.

A luminal cavity closing method using the luminal cavity closing device 70 configured as described above can include the following steps.

(1) Access Step (First Step)

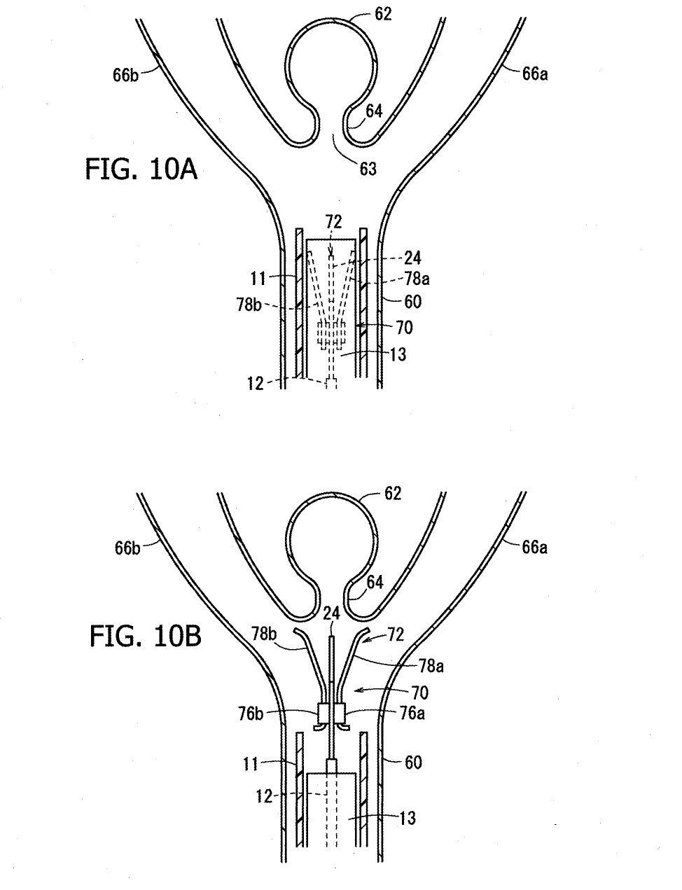

In an access step, as shown in FIGS. 10A and 10B, the luminal cavity closing device 70 is passed through the catheter 11 and is disposed in the vicinity of the luminal cavity 62. In such an access step, specifically, first, the catheter 11 with a guide wire inserted therein is fed through the blood vessel 60, and the distal end of the catheter 11 is caused to reach the blood vessel bifurcation area or the vicinity thereof. Thereafter, the guide wire is pulled out of the catheter 11. After the guide wire is pulled out of the catheter 11, the luminal cavity closing device 70 with the clamping mechanism 72 stored in the outer tube 13 is inserted into the catheter 11. In this case, as shown in FIG. 10A, the distal end of the outer tube 13 of the luminal cavity closing device 70 is located at a position slightly spaced from the luminal cavity 62 (opening 63 thereof). Next, while holding the positions of the catheter 11 and the outer tube 13, the shaft 12 is advanced in the distal direction. By this, as shown in FIG. 10B, the clamping mechanism 72 is caused to protrude from the outer tube 13, and is disposed at such a position in the blood vessel bifurcation area as to face the opening 63 of the luminal cavity 62.

(2) Insertion Step (Second Step)

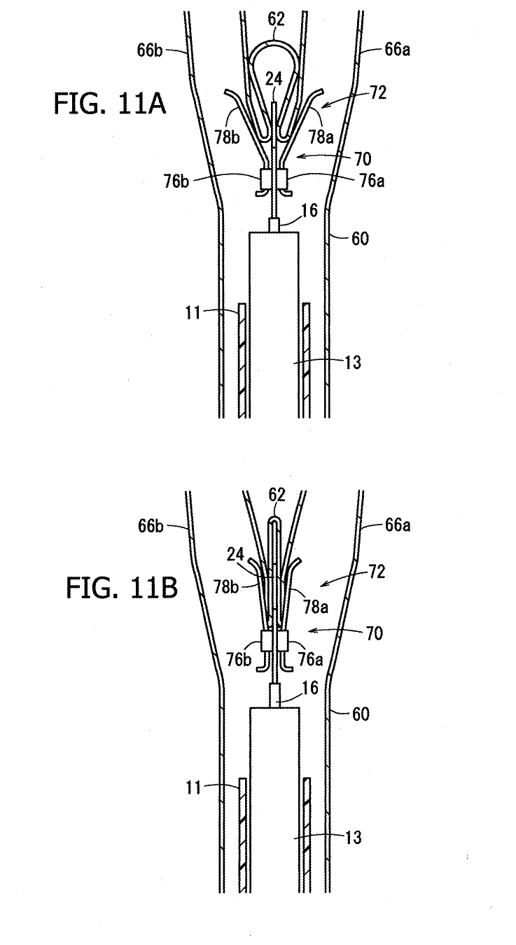

Subsequently, as shown in FIG. 11A, while holding the position of the catheter 11, the shaft 12 is advanced in the distal direction together with the outer tube 13. By this operation, the arms 78a and 78b of the clamping mechanism 72 are inserted respectively into the blood vessels 66a and 66b adjacent to and on both sides of the luminal cavity 62. In this insertion process, the blood vessels 66a and 66b adjacent to and on both sides of the luminal cavity 62 are pressed inward (toward the luminal cavity) by the inserted arms 78a and 78b. Thus, the luminal cavity 62 present between the blood vessels 66a and 66b is also pressed, whereby the internal volume of the luminal cavity 62 is reduced to a certain extent. In addition, in this embodiment, the support element 24 is provided between the arms 78a and 78b. As the arms 78a and 78b are inserted into the blood vessels, therefore, the support element 24 is inserted through the opening 63 into the luminal cavity 62.

(3) Pressing Step (Third Step)

Next, as shown in FIG. 11B, the arms 78a and 78b are closed under an action of the clamping mechanism 72, whereby the luminal cavity 62 is further pressed through the blood vessels 66a and 66b adjacent to and on both sides of the luminal cavity 62. Specifically, the arms 78a and 78b are inserted into the blood vessels 66a and 66b until it becomes impossible (or difficult) to insert the arms 78a and 78b further, because of abutment of the arms 78a and 78b on the blood vessels 66a and 66b. Thereafter, the shaft 12 is moved in the distal direction. As a result, the shape-restraining sections 76a and 76b are moved in the distal direction relative to the arms 78a and 78b. Therefore, the arms 78a and 78b whose intermediate portions have been bent in the natural state are forcibly elastically deformed by the shape-restraining sections 76a and 76b. The arms 78a and 78b thus forcibly elastically deformed by the shape-restraining sections 76a and 76b are substantially immobilized in situ. As a result of the forced elastic deformation, the interval of the arms 78a and 78b is reduced, whereby the luminal cavity 62 is pressed and contracted through the blood vessels 66a and 66b adjacent to and on both sides of the luminal cavity 62. Consequently, the opening 63 of the luminal cavity 62 is contracted or closed, and the internal volume of the luminal cavity 62 is remarkably reduced as compared with the internal volume of the luminal cavity 62 before the procedure (in the state of FIG. 10A).

(4) Detachment Step (Fourth Step)

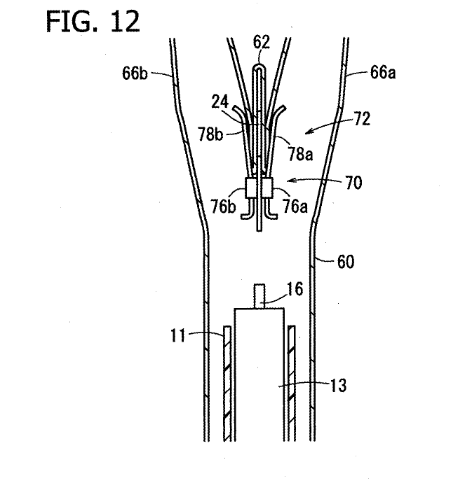

Subsequently, as shown in FIG. 12, the clamping mechanism 72 in the state of pressing the luminal cavity 62 is detached from the shaft 12 under an action of the detachment mechanism 16. The operation for detaching the detachment mechanism 72 from the shaft 12 depends on the configuration of the detachment mechanism 16, like in the detachment step(s) in the luminal cavity closing method using the luminal cavity closing device 10 as described above.

According to the luminal cavity closing device 70 in this embodiment, it is possible to carry out a procedure (luminal cavity closing method) wherein the luminal cavity closing device 70 is inserted through the catheter 11 to make access to the luminal cavity 62 generated in a blood vessel, the arms 78a and 78b are inserted into the blood vessels 66a and 66b adjacent to and on both sides of the luminal cavity 62, then the arms 78a and 78b are closed under the action of the clamping mechanism 72 to thereby press and contract the luminal cavity 62 through the blood vessels 66a and 66b adjacent to and on both sides of the luminal cavity 62, and thereafter the clamping mechanism 72 is detached from the shaft 12.

Therefore, like the luminal cavity closing device 10, the luminal cavity closing device 70 in this embodiment makes it possible to contract or close the opening 63 to thereby restrain or prevent the internal pressure of the luminal cavity 62 from rising, to effectively reduce or eliminate the danger of rupture of the luminal cavity 62, to reduce the internal volume of the luminal cavity 62, and thereby to reduce the influence of the luminal cavity 62 on the surrounding tissues.

In this embodiment, when the shape-restraining sections 76a and 76b are located in the first positions on the arms 78a and 78b, the clamping mechanism 72 assumes the expanded state. On the other hand, when the shape-restraining sections 76a and 76b are located in the second positions on the distal side of the first positions on the arms 78a and 78b, the shape-restraining sections 76a and 76b deform the arms 78a and 78b so that the clamping mechanism 72 assumes the contracted state. Therefore, the arms 78a and 78b can be closed by a simple operation in which after the arms 78a and 78b are inserted into the blood vessels 66a and 66b adjacent to and on both sides of the luminal cavity 62, the luminal cavity closing device 70 is pushed in further toward the luminal cavity 62. Accordingly, the luminal cavity closing device 70 is excellent in operability (maneuverability), and makes it possible to rapidly and easily press the luminal cavity 62 through the blood vessels 66a and 66b adjacent to and on both sides of the luminal cavity 62.

In this embodiment of FIG. 8, the same components as those components in the embodiments shown in FIGS. 1-7, naturally, offer operations and effects which are the same as or similar to the operations and effects offered by those components in the embodiments of FIGS. 1-7.

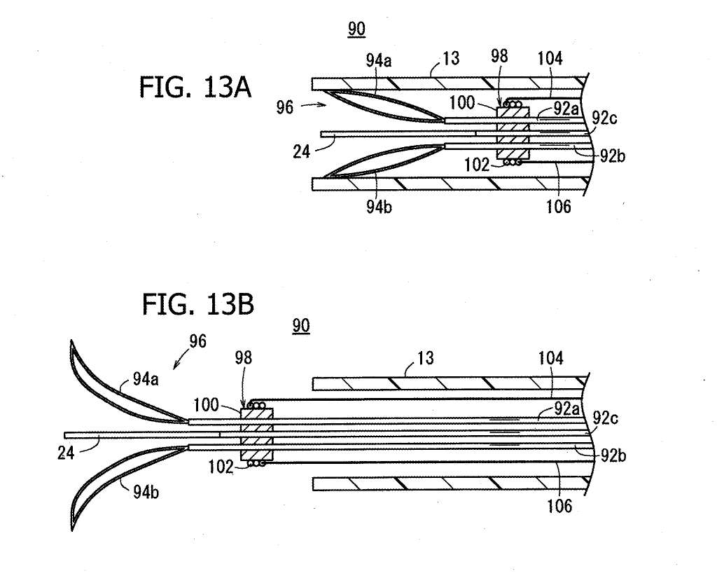

FIGS. 13A and 13B are partial schematic side views of a luminal cavity closing device 90 according to yet another embodiment of the presently disclosed subject matter. In the luminal cavity closing device 90 in this embodiment, elements exhibiting functions and effects the same as or similar to those of the elements of the luminal cavity closing device 10 are denoted by the same reference symbols as used above, and detailed descriptions of them will be omitted.

The luminal cavity closing device 90 according to this embodiment can include: an outer tube 13; plural (in the example shown, three) shafts 92a, 92b and 92c inserted in the outer tube 13; a clamping mechanism 96 provided at distal portions of the plural shafts 92a, 92b and 92c and having plural (in the example shown, two) arms 94a and 94b; and a detachment mechanism 98 for detaching the clamping mechanism 96 from the shafts 92a to 92c. The outer tube 13 can be the same in configuration as the outer tube 13 shown in FIG. 1 and the like. The grip section 18 is provided at the proximal end of the outer tube 13.

Hereinafter, the shaft 92a, the shaft 92b, and the shaft 92c will be referred to respectively as “first shaft 92a,” “second shaft 92b,” and “third shaft 92c.” The first to third shafts 92a to 92c are inserted in the outer tube 13 so that they can be displaced in the axial direction. In this embodiment, the first shaft 92a is provided at its distal end with the arm 94a on one side, while the second shaft 92b is provided at its distal end with the arm 94b on the other side. The first shaft 92a and the second shaft 92b can be rotated respectively about their axis, independently from each other. On the proximal side of the luminal cavity closing device 90, an operating section or sections (not shown) are provided for rotating the first shaft 92a and the second shaft 92b. The third shaft 92c is provided at its distal end with a support element 24 which can be the same in configuration as the support element 24 shown in FIG. 1 and the like.

The arms 94a and 94b of the clamping mechanism 96 are elastically deformable, and are formed to be curved in a natural state (in a state of being not deformed elastically). In this embodiment, the arms 94a and 94b are composed of looped wires. The interval between the distal ends of the arms 94a and 94b in the state shown in FIG. 13B may be greater than the inside diameter of the outer tube 13. In this case, as shown in FIG. 13A, the clamping mechanism 96 before being caused to protrude from the outer tube 13 is stored in the outer tube 13 in the state of being restrained from expansion by the outer tube 13 (the state of being contracted by elastic deformation). With the arms 94a and 94b composed of the looped wires, they can be stored in the outer tube 13 in the state of being radially reduced. Therefore, the outer tube 13 can be made thin (small in diameter), which contributes to enhancement of passability of the luminal cavity closing device 90 in a lumen.

Incidentally, the form of the arms 94a and 94b is not restricted to the looped wires. The arms 94a and 94b may each be composed of a single wire or plate-shaped member or other structure that can effectively achieve the functional requirements of the device.

The disengagement mechanism 98 can include: a bundling member 100 which is secured to the third shaft 92c in the vicinity of a distal portion of the third shaft 92c and through which the first shaft 92a and the second shaft 92b are inserted; a heater 102 wound in a coil form around the outer circumference of the bundling member 100; and first and second lead wires 104 and 106 connected to the heater 102. The bundling member 100 is made of a material capable of rupture by melting when heated (for example, a resin, a metal having a low melting-point, or the like). The respective outer circumferential surfaces of the first shaft 92a and the second shaft 92b and the inner circumferential surfaces of two through-holes formed in the bundling member 100 along the axial direction are in such contact with each other as not to hamper rotation of the first shaft 92a and the second shaft 92b.

The first lead wire 104 is connected to one end of the heater 102, while the second lead wire 106 is connected to the other end of the heater 102. The first and second lead wires 104 and 106 are inserted in the outer tube 13, and are connected to a power supply at their ends on the opposite side to the side of connection with the heater 102. The power supply may be either DC or AC. The detachment mechanism 98 thus configured ensures that by energizing the heater 102 through the first and second lead wires 104 and 106, the bundling member 100 (along with the first to third shafts 92a to 92c) can be put into a ruptured state by melting. Thus, the clamping mechanism 96 can be detached from the first to third shafts 92a to 92c.

An example of a luminal cavity closing method using the luminal cavity closing device 90 configured as described above can include the following steps.

(1) Access Step (First Step)

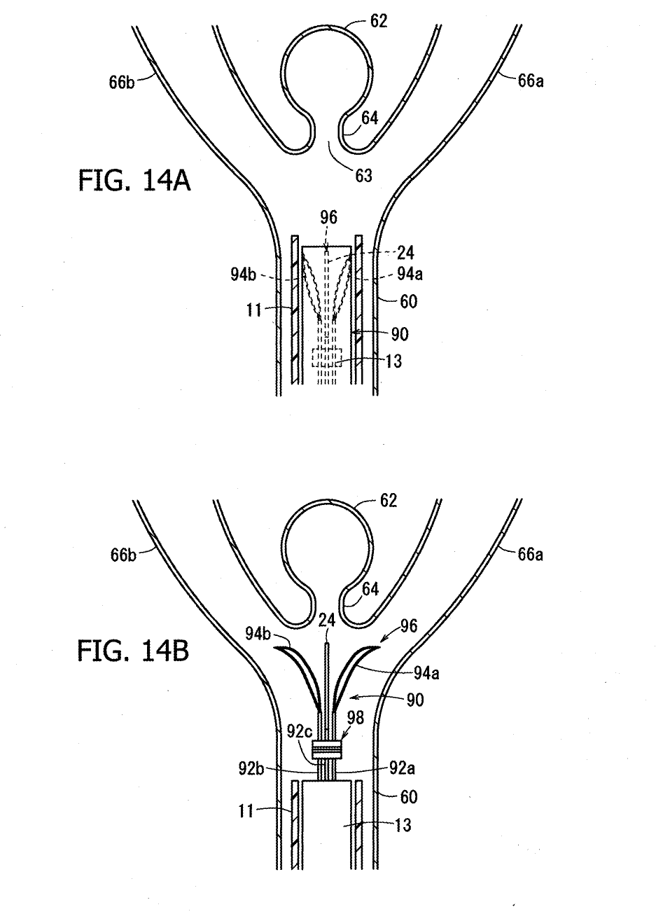

In an access step, as shown in FIGS. 14A and 14B, the luminal cavity closing device 90 is passed through the catheter 11, and is disposed in the vicinity of a luminal cavity 62. In this access step, specifically, first, the catheter 11 with a guide wire inserted therein is fed through the blood vessel 60, and the distal end of the catheter 11 is brought to a blood vessel bifurcation area or the vicinity thereof. Thereafter, the guide wire is pulled out of the catheter 11. After the guide wire is pulled out of the catheter 11, the luminal cavity closing device 90 with the clamping mechanism 96 stored in the outer tube 13 is inserted into the catheter 11. In this case, as shown in FIG. 14A, the distal end of the outer tube 13 of the luminal cavity closing device 90 is located at a position slightly spaced from the luminal cavity 62. Next, while holding the positions of the catheter 11 and the outer tube 13, the first to third shafts 92a to 92c are advanced in the distal direction. By this operation, as shown in FIG. 14B, the clamping mechanism 96 is caused to protrude from the outer tube 13, and is then disposed at such a position in the blood vessel bifurcation area as to face the opening 63 of the luminal cavity 62. The orientations of the arms 94a and 94b are set such that the arms 94a and 94b are curved outward. In other words, the arms 94a and 94b are set in the state in which the interval between them has been enlarged.

(2) Insertion Step (Second Step)

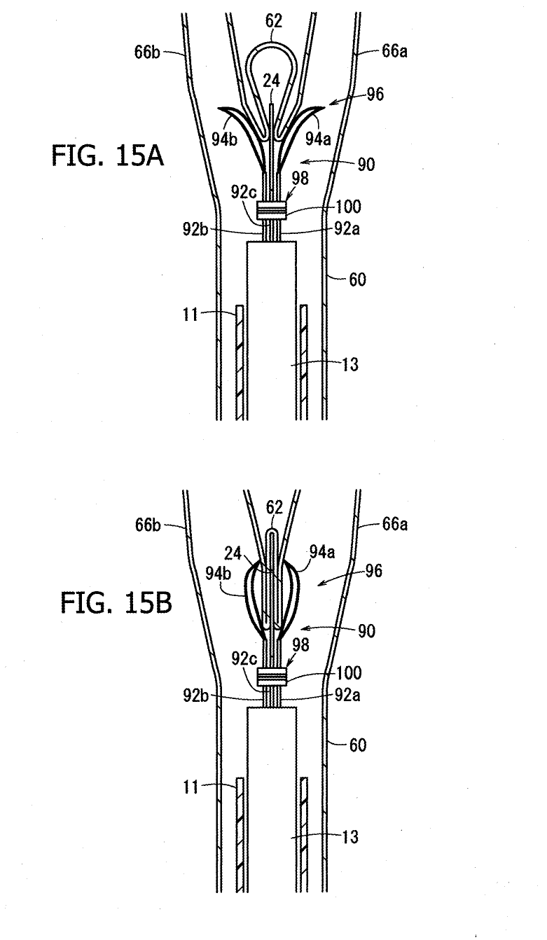

Subsequently, as shown in FIG. 15A, while holding the position of the catheter 11, the shaft 12 is advanced in the distal direction together with the outer tube 13. By this action, the arms 94a and 94b of the clamping mechanism 96 are inserted into the blood vessels 66a and 66b adjacent to and on both sides of the luminal cavity 62. In this insertion process, the blood vessels 66a and 66b adjacent to and on both sides of the luminal cavity 62 are pressed inward (toward the luminal cavity) by the arms 94a and 94b inserted therein. In addition, the luminal cavity 62 present between these blood vessels is also pressed, whereby the internal volume of the luminal cavity 62 is reduced to a certain extent. In this embodiment, the support element 24 is provided between the arms 94a and 94b. Therefore, as the arms 94a and 94b are inserted into the blood vessels, the support element 24 is inserted via the opening 63 into the luminal cavity 62.

(3) Pressing Step (Third Step)

Next, as shown in FIG. 15B, the arms 94a and 94b are closed under an action of the clamping mechanism 96, whereby the luminal cavity 62 is further pressed through the blood vessels 66a and 66b adjacent to and on both sides of the luminal cavity 62. Specifically, the first shaft 92a and the second shaft 92b are each rotated respectively about its axis, whereby the orientations of the arms 94a and 94b are so changed as to reduce the interval between the arms 94a and 94b. In other words, the orientations are so changed that the arms 94a and 94b having been curved outward become curved inward. Thus, the luminal cavity 62 is pressed and contracted through the blood vessels adjacent to and on both sides of the luminal cavity 62. Consequently, the opening 63 of the luminal cavity 62 is contracted or closed, whereby the internal volume of the luminal cavity 62 is markedly reduced as compared with the internal volume of the luminal cavity 62 before the procedure (in the state of FIG. 14A).

(4) Detachment Step (Fourth Step)

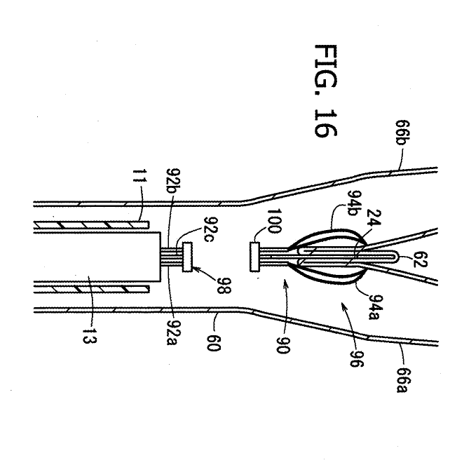

Subsequently, as shown in FIG. 16, the clamping mechanism 96, when in the state of pressing the luminal cavity 62, is detached from the first to third shafts 92a to 92c under an action of the detachment mechanism 98. Specifically, the heater 102 is energized to cause fracture by melting (fusing) of the bundling member 100 together with the first to third shafts 92a to 92c, whereby the detachment mechanism 98 is detached from the first to third shafts 92a to 92c. In this case, distal end portions of the first to third shafts 92a to 92c having undergone rupture (by melting) together with the bundling member 100 are fused to a part of the bundling member 100 also having undergone rupture (by melting). Therefore, the two arms 94a and 94b and the support element 24 are united, so that their positional relationship is maintained.

According to the luminal cavity closing device 90 in this embodiment, it is possible to carry out a procedure (luminal cavity closing method) wherein the luminal cavity closing device 90 is inserted through the catheter 11 to make access to a luminal cavity 62 in a living body, the arms 94a and 94ba are inserted respectively into the blood vessels 66a and 66b adjacent to and on both sides of the luminal cavity 62, then the arms 94a and 94b are closed under the action of the clamping mechanism 96 so as to press and contract the luminal cavity 62 through the blood vessels 66a and 66b adjacent to and on both sides of the luminal cavity 62, and thereafter the clamping mechanism 96 in the state of pressing the luminal cavity 62 is detached from the shaft 12.

Therefore, like the luminal cavity closing devices 10 and 70 according to the embodiments previously described above, the luminal cavity closing device 90 of this embodiment also makes it possible to contact or close the opening 63 so as to restrain or prevent the internal pressure of the luminal cavity 62 from rising, to effectively eliminate or reduce the danger of rupture of the luminal cavity 62, to reduce the internal volume of the luminal cavity 62, and thereby to reduce the influence of the luminal cavity 62 on the surrounding tissues.

In addition, in this embodiment, by rotating the first and second shafts 92a and 92b, the orientations of the arms 94a and 94b are changed so as to thereby contract the clamping mechanism 96. Therefore, the mechanism which operates so as to clamp (pinch) the luminal cavity 62 through the blood vessels 66a and 66b adjacent to and on both sides of the luminal cavity 62 can be realized with a simple configuration.

Furthermore, in this embodiment, when the bundling member 100 is melted under heating by the heater 102, the first to third shafts 92a to 92c are melted together with the bundling member 100 in the melted area. This ensures that even after the disengagement of the disengagement mechanism 98 from the first to third shafts 92a to 92c, the arms 94a to 94c would not be separated away. Consequently, the state in which the luminal cavity 62 is pressed by the arms 94a and 94b can be assuredly and stably maintained.

In this embodiment, the same components as those components in the previously described embodiments, naturally, offer operations and effects which are the same as or similar to the operations and effects offered by those components of the previous embodiments.

While the presently disclosed subject matter has been described by showing and describing exemplary embodiments, the invention is not to be restricted to the above embodiments, and various alterations are naturally possible within the scope of the gist of the invention.

What is claimed is:

1. A luminal cavity closing device comprising:
a flexible shaft;
a clamping mechanism located at a distal portion of the shaft, the clamping mechanism including at least two arms configured to be inserted into lumens of hollow tubular structures located adjacent to a luminal cavity present in a hollow tubular structure bifurcation area, the clamping mechanism being operable between an expanded state where an interval distance between the arms is enlarged to a contracted state where the interval distance between the arms is reduced relative to the expanded state; and
a detachment mechanism configured to detach the clamping mechanism from the shaft.

2. The luminal cavity closing device according to claim 1, further comprising
an expansion-releasing section configured to change from a first state in which the expansion-releasing section is present between the arms so as to enlarge the interval distance between the arms against elastic forces to a second state in which the expansion-releasing section allows for release of forced expansion of the arms.

3. The luminal cavity closing device according to claim 1,
wherein the arms are each bent or curved at least at an intermediate portion of a longitudinal axis of each of the arms;
the clamping mechanism includes a base part connected to the shaft, and a shape-restraining section located at the base part and through which the arms extend; and
when the shape-restraining section is in a first position on the arms, the clamping mechanism assumes the expanded state, whereas when the shape-restraining section is in a second position on a distal side of the first position, the shape-restraining section deforms the arms to cause the clamping mechanism to assume the contracted state.

4. The luminal cavity closing device according to claim 1, further comprising:
at least one additional shaft, wherein the shaft and the additional shaft are each configured to be rotated about a respective axis thereof,
wherein each of the arms is curved and located on a distal end of a respective one of the shaft and additional shaft, and
an orientation of each of the arms is changed with rotation of each of the shaft and the additional shaft, respectively, whereby the clamping mechanism is caused to assume the contracted state via the rotation.

5. The luminal cavity closing device according to claim 4,
wherein the detachment mechanism includes a bundling member through which the shaft and the additional shaft are slidably located, and a heater configured to melt the bundling member during operation; and
when the bundling member is melted during operation of the heater, the shaft and the additional shaft are fused together with the bundling member in a melting area, whereby the clamping mechanism is detached from the shaft and the additional shaft.

6. The luminal cavity closing device according to claim 1,
wherein a support element configured for insertion into the luminal cavity is located between the arms.

7. The luminal cavity closing device of claim 1, wherein the flexible shaft is a linear shaft.

8. The luminal cavity closing device according to claim 1, further comprising:
an expansion-releasing section located between the arms such that the arms are elastically deformed away from each other.

9. The luminal cavity closing device according to claim 8, further comprising
a wire connected to the expansion-releasing section and configured to move the expansion-releasing section such that the arms are allowed to elastically return to a position closer to each other after the expansion-releasing section is moved.

10. The luminal cavity closing device according to claim 1, wherein the detachment mechanism includes at least one of the following:
a heater;
a male/female fitting;
a hook and engagement structure; and
a ball and holding ring structure.

11. A luminal cavity closing method comprising:
providing a luminal cavity closing device including a flexible shaft, a clamping mechanism located at a distal end of the shaft and having at least two arms, and a detachment mechanism configured to detach the clamping mechanism from the shaft;
passing the luminal cavity closing device through a catheter inserted in a hollow tubular structure and advancing the luminal cavity closing device to a position where a luminal cavity formed in a hollow tubular structure bifurcation area is present;
inserting each of the arms into a respective hollow tubular structure located adjacent to the luminal cavity;
reducing an interval distance between the arms of the clamping mechanism so as to press the luminal cavity through the hollow tubular structures located adjacent to the luminal cavity; and
detaching the clamping mechanism while the clamping mechanism is in a state of clamping the luminal cavity.

12. The luminal cavity closing method according to claim 11,
wherein inserting includes inserting a support element provided between the arms into the luminal cavity.

13. The luminal cavity closing method according to claim 11, wherein the tubular structures are located on opposite sides of the luminal cavity such that the clamping state is a configuration in which the tubular structures press the luminal cavity from opposing sides of the luminal cavity.

14. The luminal cavity closing method according to claim 11, wherein reducing an interval distance between the arms of the clamping mechanism includes rotating the shaft about a longitudinal axis of the shaft.

15. The luminal cavity closing method according to claim 11, wherein reducing an interval distance between the arms of the clamping mechanism includes moving a wire located adjacent and extending parallel with the shaft.

16. The luminal cavity closing method according to claim 11, wherein reducing an interval distance between the arms of the clamping mechanism includes removing a structure from between the arms to allow the arms to elastically deform towards each other.

17. The luminal cavity closing method according to claim 16, wherein removing includes melting a structure located adjacent the arms.

18. The luminal cavity closing method according to claim 11, wherein detaching includes moving the shaft in a distal direction to unlock the clamping mechanism from the shaft.

19. The luminal cavity closing method according to claim 11, wherein detaching includes melting a structure to detach the clamping mechanism from the shaft.

20. The luminal cavity closing method according to claim 11, further comprising:
reducing a volume of the luminal cavity by moving the arms relative to each other.
